(19) United States
(12) Patent Application Publication
(10) Pub. No.: US 2018/0306682 A1
(43) Pub. Date: Oct. 25, 2018

(54) SMART PUMP FOR A PORTABLE GAS DETECTION INSTRUMENT

(71) Applicant: Gas Measurement Instruments Limited, St. Paul, MN (US)

(72) Inventors: Andrew Glendinning, Kilcreggan (GB); Ian Binns, Glasgow (GB); Fraser Mathieson, Glasgow (GB)

(21) Appl. No.: 15/771,456

(22) PCT Filed: Oct. 20, 2016
(86) PCT No.: PCT/GB2016/053279

§ 371 Date: Apr. 27, 2018

(30) Foreign Application Priority Data
Oct. 29, 2015 (GB) 1519145.5

(51) Int. Cl. G01N 1/24; G01N 1/22; G01N 33/00; G01N 1/14

(52) U.S. Cl. CPC G01N 1/24; G01N 1/14; G01N 33/0009; G01N 1/2273

(57) ABSTRACT

A diaphragm pump (1) in a gas detection instrument has an inlet (2) which is in fluid communication with a pump chamber (4), and an outlet (3) through which fluid in the pump chamber (4) can be expelled from the pump (1). A wall of the pump chamber (4) is formed by a circuit board 6 on an inner surface of which is mounted a pressure sensor (5) so as to be in fluid communication with the interior of the pump chamber 4 for detecting and monitoring the pressure of the gas passing through the pump chamber (4).

The present invention relates to pumps for portable gas detection instruments.

Portable gas detection instruments typically have a pump integrated into the instrument which is used to draw air/gas from the surrounding environment and delivery it to the gas sensors incorporated into the instrument for analysis.

In order to ensure reliable operation of the device and also to ensure early identification of issues which could affect the validity of the readings given by the instrument, it is important that the condition of the pump is monitored during operation of the device. This is typically done by use of a flow fail detector which monitors the gas flow rate and uses variances in the flow rate outside of defined thresholds to be indicative of a problem. For example, the pump inlet may become blocked or the instrument may inadvertently suck in liquid, both of which would result in a reduction in the flow rate through the instrument. Upon detecting such an occurrence, the flow fail detector operates to switch off the pump at the earliest opportunity to limit damage to it.

The fail flow detector normally comprises a pressure sensor. Conventionally, this is located within the instrument in the flow path between the pump and the instrument inlet so that the gas pressure is monitored as it travels towards or away from the pump. The pressure sensor is connected to a microprocessor in the instrument which analyses the readings therefrom. This prior art locating of the pressure sensor, however, has the drawback that it imposes limitations on the design of the instrument flow path due to the need to accommodate the pressure sensor therein. It also increases the complexity of the supporting circuitry and layout. Furthermore, it has been found that the flow rate between pumps of the same design can vary significantly. The flow rate of the instrument is set by supplying a certain voltage to the pump, which will normally be fixed during the life of the pump. The supply voltage is normally set at the same level for every pump of the same design. In practice, however, it is found that pumps configured with the same supply voltage actually produce differing flow rates, and existing systems do not allow an easy way to check for and correct such variance.

According to a first aspect of the present invention there is provided a pump for a gas detection instrument having an inlet, an outlet and a pump chamber located between the inlet and the outlet, the pump further comprising at least one sensor mounted in the pump chamber for detecting, in use, at least one parameter of a gas passing through the pump chamber.

The present invention further provides a gas detection instrument having a sampling inlet, at least one gas sensor for analysing a gas sampled through the sampling inlet and a pump for drawing a gas sample through the sampling inlet and delivering it to the at least one gas sensor, the pump having an inlet, an outlet and a pump chamber located between the inlet and the outlet, the pump further comprising at least one sensor mounted in the pump chamber for detecting, in use, at least one parameter of a gas passing through the pump chamber.

The pump according to the invention has the advantage that, by locating the sensor within the pump chamber, rather than outside the pump as with the prior art, the pump is self contained and thereby provides a modular design which can easily be calibrated, whilst, at the same time, enabling the flow path and circuitry of the remainder of the gas detection instrument to be simplified. Furthermore, the integrating of the sensor into the pump, in particular when the sensor is a flow rate sensor such as a pressure sensor, enables an intelligent control system to be implemented on the pump which enables the pump to self-regulate flow rate, rather than having a simple on/off operation, thereby eliminating variations in pump to pump performance.

Preferably, the at least one sensor is a pressure sensor. Other sensors may also be provided such as at least one of a temperature sensor and a flow rate sensor.

In a particularly preferred embodiment of the invention, the at least one sensor is mounted on a circuit board which is integrated into the pump and which, in particular forms a side wall of the pump chamber. The circuit board may then include a processor which operates in conjunction with the at least one sensor to monitor the performance of the pump and control it through an interface with a pump controller which may be separate to the processor on the circuit board or integrated into it.

The present invention further provides a method of operating a pump in a gas detection instrument comprising the steps of providing a pump in a gas detection instrument, the pump having an inlet, an outlet, a pump chamber located between the inlet and the outlet, a pump motor operable to drive the pump, and a pressure sensor mounted in the pump chamber for detecting, in use, at least one parameter of a gas passing through the pump chamber, the pressure sensor being connected to a microprocessor, reading the pressure within the pump chamber from the pressure sensor using the microprocessor, calculating the flow rate through the pump chamber using the reading from the pressure sensor, and varying a supply voltage delivered to the pump motor in order to vary the speed of the pump so as to control the flow rate through the pump to maintain the flow rate at a predefined value.

Preferably, the processor reads the pressure in the chamber before the pump starts and records this as an ambient pressure reading, and compares the pressure readings after the pump starts with the ambient reading to confirm that pressure has dropped so as to indicate flow through the pump.

In a preferred embodiment, the processor is programmed with a threshold value for the rate of drop of pressure within the chamber, the processor being programmed to shut down the pump if the measured rate of pressure drop is greater than the threshold value as being indicative of a blockage in the sample line.

In order that the invention may be well understood, there will now be described an embodiment thereof, given by way of example, reference being made to the accompanying drawings, in which:

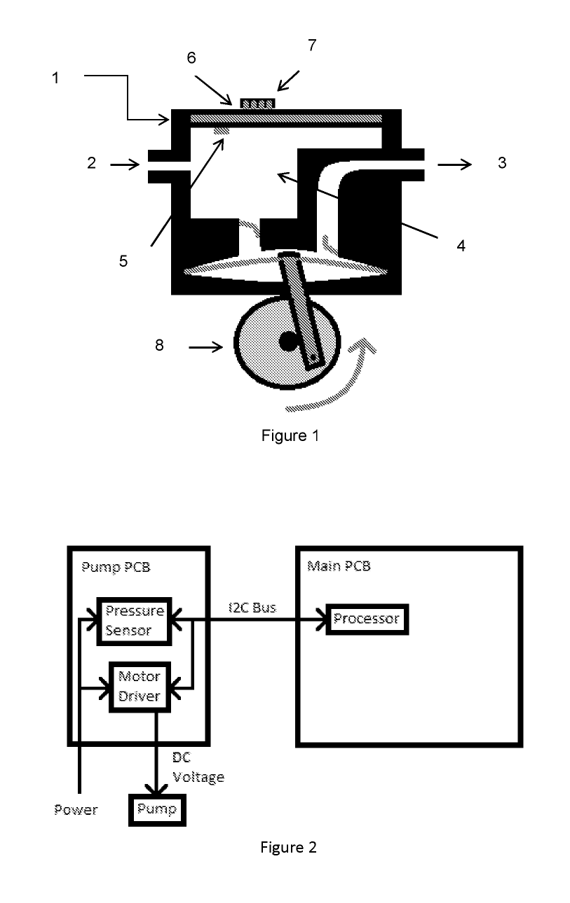

FIG. 1 is a diagrammatic illustration of a diaphragm pump with a sensor incorporated into the pump chamber according to the present invention; and

FIG. 2 is a block diagram of a pump of the invention with integrated pressure sensor and motor.

Referring to FIG. 1, there is shown a diaphragm pump 1 as an example of a pump for a gas detection instrument of the type of the present invention. The pump 1 has an inlet 2 which is in fluid communication with a pump chamber 4, and an outlet 3, which is also in fluid communication with the pump chamber 4, such that operation of the pump 1 causes gas to be drawn into the pump chamber 4 through the inlet 2 and to be expelled through the outlet 3. The pump shown in FIG. 1 is a diaphragm pump of the type well known in the art, but it will be understood that the invention is not limited to that type of pump and is applicable to any pump which may be used for drawings gas into a gas detection instrument.

As can clearly be seen in FIG. 1, the upper wall of the pump chamber 4 is formed by a printed circuit board (PCB) 6 which is suitably finished on the bottom to ensure a fluid tight connection with the neighbouring walls of the pump chamber 4 to avoid fluid leaking from the pump chamber 4 around or through the PCB.

A pressure sensor 5 is mounted on the inner surface of the PCB 6 which faces into the pump chamber 4 so that the pressure sensor 5 is in fluid communication with the interior of the pump chamber 4 and is therefore responsive to the pressure of gas passing through the pump chamber 4. A microprocessor 7 is mounted on the upper surface of the PCB 6 and is in communication with the pressure sensor 5 so as to be able to process the output of the pressure sensor. The connection between the pressure sensor and the microprocessor may be a wired connection, which also enables power to be delivered to the pressure sensor, or may be a wireless communication system. The pressure sensor may also have its own power source included within the pressure chamber 4 or the pressure sensor may be a passive device, such as a SAW based sensor which does not require a power supply. The pressure sensor itself may be any suitable sensor well known in the art for sensing gas pressure.

In operation, the pump is operated by power being delivered to a pump motor by a main processor 10 as shown in FIG. 2, which causes gas to be drawn through the inlet 2, into the pump chamber 4, then expelled out through the outlet 3. The pressure sensor 5 detects the pressure of the gas within the pump chamber 4 and the microprocessor 7 uses the output of the pressure sensor 5 to calculate the flow rate of gas through the pump in a manner well known in the art.

In the preferred embodiment shown in FIG. 2, the PCB 6 also includes a motor drive 11 which is connected to the pump motor 8 for controlling the speed of the motor. The pump processor 7 is also connected to a main processor 10. The pump processor 7 reads the pressure from the pressure sensor 5 and also sets the drive voltage to the pump so ensure correct flow rate through the pump. At power up, before the pump starts an ambient pressure reading is taken from the pressure sensor 5. When the pump starts, the pressure will fall due to pressure drop from filters, sample line etc., and this provides and indication of flow through the pump.

If the sample line is blocked, the pressure will fall rapidly. The processor senses this and turns the pump off.

Although in FIG. 2 the main processor 10 is shown on a separate circuit board, it will be understood that the main processor main instead be mounted on the pump PCB 6 for a more compact configuration. This will also allow faster response, calibration of actual components and reduced load on the main processor.

It is also possible to include additional environmental sensors either inside and/or outside of the pump chamber, which communicate with the pump processor and/or the main processor for further monitoring of the pump status. For example, a temperature sensor may be used to monitor the temperature of gas in the pump chamber as well as for monitoring environmental temperature

1. A pump for a gas detection instrument having an inlet, an outlet and a pump chamber located between the inlet and the outlet, the pump further comprising at least one sensor mounted in the pump chamber for detecting, in use, at least one parameter of a gas passing through the pump chamber.

2. A pump according to claim 1, wherein the at least one sensor includes a pressure sensor.

3. A pump according to claim 1, wherein the at least one sensor includes at least one of a temperature sensor and a flow rate sensor.

4. A pump according to claim 1, wherein the at least one sensor is mounted on a circuit board which is integrated into the pump.

5. A pump according to claim 4, wherein the circuit board forms a side wall of the pump chamber.

6. A pump according to claim 4, wherein the circuit board includes a processor which operates in conjunction with the at least one sensor to monitor the performance of the pump and control it through an interface with a pump controller.

7. A pump according to claim 6, wherein the pump controller is separate to the processor on the circuit board.

8. A pump according to claim 6, wherein the pump controller is integrated into the processor on the circuit board.

9. A gas detection instrument having a sampling inlet, at least one gas sensor for analysing a gas sampled through the sampling inlet and a pump according to claim 1 for drawing a gas sample through the sampling inlet and delivering it to the at least one gas sensor.

10. A method of operating a pump in a gas detection instrument comprising the steps of providing a pump according to claim 1 in a gas detection instrument, the pressure sensor in the pump being connected to a microprocessor, reading the pressure within the pump chamber from the pressure sensor using the microprocessor, calculating the flow rate through the pump chamber using the reading from the pressure sensor, and varying a supply voltage delivered to the pump motor in order to vary the speed of the pump so as to control the flow rate through the pump to maintain the flow rate at a predefined value.

11. A method according to claim 10, comprising the further step of the processor reading the pressure in the chamber before the pump starts and recording this as an ambient pressure reading, and comparing the pressure readings after the pump starts with the ambient reading to confirm that pressure has dropped so as to indicate flow through the pump.

12. A method according to claim 10, wherein the processor is programmed with a threshold value for the rate of drop of pressure within the chamber, the processor being programmed to shut down the pump if the measured rate of pressure drop is greater than the threshold value as being indicative of a blockage in the sample line.
